(19) United States
(12) Patent Application Publication
(10) Pub. No.: US 2014/0271861 A1
(43) Pub. Date: Sep. 18, 2014

(54) MATRIX COMPOSITIONS FOR CONTROLLED RELEASE OF PEPTIDE AND POLYPEPTIDE MOLECULES

(71) Applicant: POLYPID LTD., Petach Tikva (IL)

(72) Inventor: Noam Emanuel, Rehovot (IL)

(21) Appl. No.: 14/235,075

(22) PCT Filed: Jul. 26, 2012
(86) PCT No.: PCT/IL2012/050278

§ 371 Date: Jan. 26, 2014

Related U.S. Application Data
(60) Provisional application No. 61/512,025, filed on Jul. 27, 2011.

(51) Int. Cl. A61K 9/70; A61K 38/18; A61K 38/10

(52) U.S. Cl. CPC A61K 9/70; A61K 38/10; A61K 38/1825
USPC 424/484; 514/1.1; 514/2.3

(57) ABSTRACT

The present invention provides compositions for controlled release of a peptidic molecule comprising a lipid-saturated matrix comprising a biocompatible polymer and a peptidic molecule associated with PEG. The present invention also provides methods of producing the matrix compositions and methods for using the matrix compositions to provide controlled release of the peptidic molecule.

### FIELD OF THE INVENTION

The present invention provides compositions for controlled release of a peptidic molecule comprising a lipid-saturated matrix comprising a biocompatible polymer and a peptidic molecule associated with PEG. The present invention also provides methods of producing the matrix compositions and methods for using the matrix compositions to provide controlled release of a peptidic active molecule.

### BACKGROUND OF THE INVENTION

The potential therapeutic or diagnostic effects of various peptides or proteins have been intensively studied during the last decades, and a variety of diseases and clinical disorders are treated by the administration of such pharmaceutically active agents. A technological barrier to the use of peptidic molecule, however, is the need for practical, effective and safe means for their delivery and sustained and/or controlled release.

Lipid based delivery systems for biologically active agents, particularly therapeutic agents are well known in the art of pharmaceutical science. Typically they are used to formulate agents having poor bioavailability or high toxicity or both. Among the prevalent dosage forms that have gained acceptance are many different types of liposomes, including small unilamellar vesicles, multilamellar vesicles and many other types of liposomes; different types of emulsions, including water in oil emulsions, oil in water emulsions, water-in-oil-in-water double emulsions, submicron emulsions, microemulsions; micelles and many other hydrophobic drug carriers. These types of lipid based delivery systems can be highly specialized to permit targeted delivery or decreased toxicity or increased metabolic stability and the like. Extended release of the biologically active agent in the range of days, weeks and more are not profiles commonly associated with lipid based delivery systems in vivo.

Ideally sustained release drug delivery systems should exhibit kinetic and other characteristics readily controlled by the types and ratios of the specific excipients used. Advantageously the sustained release drug delivery systems should provide solutions for hydrophilic, amphipathic as well as hydrophobic drugs.

It has been long appreciated that administration of a therapeutic agent in a manner that does not afford controlled release may lead to substantial oscillation of its levels, at times reaching concentrations that could be toxic or produce undesirable side effects, and at other times falling below the levels required for therapeutic efficacy. A primary goal of the use of devices and/or methods for controlled release is to produce greater control over the systemic levels of therapeutic agents.

Various strategies have been developed aiming at achieving controlled release of a therapeutic agent. Release by controlled diffusion is one of these strategies. Different materials have been used to fabricate diffusion-controlled slow release devices. These materials include non-degradable polymers such as polydimethyl siloxane, ethylene-vinyl acetate copolymers, and hydroxylalkyl methacrylates as well as degradable polymers, among them lactic/glycolic acid copolymers. Microporous membranes fabricated from ethylene-vinyl acetate copolymers have been used for release of proteins, affording a high release capacity.

An additional strategy for controlled release involves chemically controlled sustained release, which requires chemical cleavage from a substrate to which a therapeutic agent is immobilized, and/or biodegradation of the polymer to which the agent is immobilized. This category also includes controlled non-covalent dissociation, which relates to release resulting from dissociation of an agent, which is temporarily bound to a substrate by non-covalent binding. This method is particularly well suited for controlled release of proteins or peptides, which are macromolecules capable of forming multiple non covalent, ionic, hydrophobic, and/or hydrogen bonds that afford stable but not permanent attachment of proteins to a suitable substrate.

Ideally sustained release drug delivery systems should exhibit kinetic and other characteristics readily controlled by the types and ratios of the specific excipients used. Advantageously the sustained release drug delivery systems should provide solutions for hydrophilic, amphipathic as well as hydrophobic drugs.

International Patent Application Publication Nos. WO 2010/007623 and WO 2011/0072525 to the inventors of the present invention provides compositions for extended release of one or more active ingredients, comprising a lipid-saturated matrix formed from a biodegradable, non-biodegradable or a block-co-polymers comprising a non-biodegradable polymer and a biodegradable polymer. Methods of producing the matrix compositions and methods for using the matrix compositions to provide controlled release of an active ingredient in the body of a subject in need thereof are also disclosed.

Despite the advances recently made in the art, there is a need for improved pharmaceutical compositions adapted to achieve sustained release or programmed release or controlled release from a lipid-saturated polymeric matrix of multiple pharmaceutically active agents, preferably in combination with immediate release of the same or additional active agents.

### SUMMARY OF THE INVENTION

The present invention provides compositions for controlled release of a peptidic molecule comprising a lipid-saturated matrix comprising a biocompatible polymer and a peptidic molecule associated with PEG. The matrix composition is particularly suitable for local delivery or local application of the peptidic molecule. The present invention also provides methods of producing the matrix compositions and methods for using the matrix compositions to provide controlled and/or sustained release of a biologically active peptidic molecule.

The present invention is based in part on the unexpected discovery that peptides, polypeptides or proteins and in particular polar peptidic molecules present in organic solvent solutions that further comprise polyethylene glycol (PEG) can be efficiently loaded into a lipid-based matrix comprising at least one biocompatible polymer, wherein the polymer can be biodegradable polymer, non-biodegradable polymer or a combination thereof. Furthermore, the peptidic molecule can be released from the matrix in a controlled and/or extended manner.

The matrix compositions of the present invention is advantageous over hitherto known compositions and matrices for the delivery of a biologically active peptidic molecule in that it combines efficient local delivery of the biologically active molecule to cells or tissues with controlled and/or sustained release of said molecule.

In one aspect, the present invention provides a matrix composition comprising: (a) a pharmaceutically acceptable biocompatible polymer in association with a first lipid component comprising at least one lipid having a polar group; (b) a second lipid component comprising at least one phospholipid having fatty acid moieties of at least 14 carbons; (c) at least one peptidic molecule and in association with polyethylene glycol (PEG), wherein the matrix composition is adapted for providing sustained and/or controlled release of the peptidic molecule. According to some embodiments, the peptidic molecule is polar. According to some embodiments, the peptidic molecule is hydrophilic.

According to certain currently typical embodiments, the polymer and the phospholipids form a matrix composition that is substantially free of water.

The term “peptidic molecule” as used herein refers to any structure comprised of one or more amino acids, typically of two or more amino acids. The term intends to include peptides, polypeptides and proteins. The peptidic molecule can be a naturally occurring peptide, polypeptide or protein, a modified, a recombinant or a chemically synthesized peptide, polypeptide or protein.

The term “polar” in conjunction with the peptidic molecule as defined above means that the peptidic molecule comprises at least one amino acid having a polar functional group. For example, cationic side chains (arginine and lysine), anionic side chains (aspartate and glutamate), and neutral polar side chains (asparagine, glutamine, serine, and threonine). According to some embodiments it means that the overall character of the molecule is polar. According to some embodiments it means that the molecule is soluble in a polar solvent.

According to certain embodiments, the peptidic molecule has a therapeutic activity. According to certain embodiments, the peptidic molecule is selected from an enzyme, a hormone, an anti-microbial agent, an antibody, an anti-cancer drug, an osteogenic factor, a growth factor or a low oral bioavailability protein or peptide. According to some embodiments, the peptidic molecule is polar. Each possibility represents a separate embodiment of the invention. According to certain typical embodiments, the peptidic molecule is an anti-microbial peptide. According to other typical embodiments, the peptidic molecule is an enzyme.

According to certain embodiments, the peptidic molecule is non-covalently associated with PEG. Without wishing to be bound by theory or mechanism of action, it is suggested that the association of the peptidic molecule and PEG is generally a product of intermolecular interactions including hydrogen bonding and the attractive action of Van der Waals forces.

According to certain embodiments, the PEG is a linear PEG having a molecular weight in the range of 1,000-10,000. According to typical embodiments, the PEG molecular weight is in the range of 1,000-8,000, more typically of 5,000 or less. Biodegradable PEG molecules, particularly PEG molecules comprising degradable spacers having higher molecular weights can be also used according to the teachings of the present invention.

PEG molecules having a molecular weight of 5,000 or less are currently approved for pharmaceutical use. Thus, according to certain typical embodiments, the active PEG molecules have a molecular weight of up to 5,000.

According to some embodiments the matrix composition may further comprise at least one cationic lipid. According to certain embodiments, the cationic lipid is selected from the group consisting of DC-Cholesterol, 1,2-dioleoyl-3-trimethylammonium-propane (DOTAP), Dimethyldioctadecylammonium (DDAB), 1,2-dilauroyl-sn-glycero-3-ethylphosphocholine (Ethyl PC), 1,2-di-O-octadecenyl-3-trimethylammonium propane (DOTMA), and others. Each possibility represents a separate embodiment of the present invention.

According to certain embodiments, the biocompatible polymer is selected from the group consisting of biodegradable polymer, non-biodegradable polymer and a combination thereof. According to certain embodiments the biodegradable polymer comprises polyester selected from the group consisting of PLA (polylactic acid), PGA (poly glycolic acid), PLGA (poly(lactic-co-glycolic acid)) and combinations thereof. According to additional embodiments, the biodegradable polymer is selected from the group consisting of chitosan and collagen. According to other embodiments, the non-biodegradable polymer is selected from the group consisting of polyethylene glycol (PEG), PEG acrylate, PEG methacrylate, methylmethacrylate, ethylmethacrylate, butylmethacrylate, 2-ethylhexylmethacrylate, laurylmethacrylate, hydroxylethyl methacrylate, 2-methacryloyloxyethylphosphorylcholine (MPC), polystyrene, derivatized polystyrene, polylysine, poly N-ethyl-4-vinyl-pyridinium bromide, poly-methylacrylate, silicone, polyoxymethylene, polyurethane, polyamides, polypropylene, polyvinyl chloride, polymethacrylic acid, and derivatives thereof alone or as co-polymeric mixtures thereof. Each possibility represents a separate embodiment of the present invention.

According to additional embodiments, the non-biodegradable polymer and the biodegradable polymer form a block co-polymer, for example, PLGA-PEG-PLGA and the like.

According to certain embodiments the lipid having a polar group is selected from the group consisting of a sterol, a tocopherol, a fatty acid, a phosphatidylethanolamine or any combination thereof. According to certain particular embodiments, the lipid having a polar group is sterol or a derivative thereof. According to typical embodiments, the sterol is cholesterol.

According to certain embodiments the first lipid component is mixed with the biocompatible polymer to form a non-covalent association. Without being limited to any particular theory or mechanism of action it is suggested that the polymer and the first lipid having a polar group are associated via the formation of hydrogen bonds.

According to certain particular embodiments, the first lipid component is sterol or a derivative thereof and the bio-compatible polymer is biodegradable polyester. According to these embodiments, the biodegradable polyester is associated with the sterol via non-covalent bonds in particular via hydrogen bonds.

According to some embodiments the second lipid component comprises a phosphatidylcholine having two fatty acid moieties wherein at least one of the fatty acid moieties is of at least 14 carbons, or a derivative thereof. According to some embodiments at least one of the fatty acid moieties is saturated. According to some embodiments both fatty acid moieties are saturated. According to other embodiments the second lipid component comprises a mixture of phosphatidylcholines having two fatty acid moieties wherein at least one of the fatty acid moieties is of at least 14 carbons, or derivatives thereof. According to some embodiments at least one of the fatty acid moieties is saturated. According to some embodiments both fatty acid moieties are saturated. According to yet other embodiments the second lipid component comprises a mixture of a phosphatidylcholine and a phosphatidylethanolamine or derivatives thereof. According to additional embodiments, the second lipid component further comprises a sterol and derivatives thereof. According to typical embodiments, the sterol is cholesterol. According to yet further embodiments the second lipid component comprises a mixture of phospholipids of various types. According to certain typical embodiments, the second lipid component further comprises at least one of a sphingolipid, a tocopherol and a pegylated lipid.

According to additional embodiments, the weight ratio of the total lipids to the biocompatible polymer is between 1:1 and 9:1 inclusive. According to some embodiments the weight ratio of the first lipid to the second lipid is between 1:20 to 1:1. According to some embodiments the weight ratio of the peptidic molecule and PEG is between 20:1 and 1:1. According to some embodiments, PEG is present in an amount of between 0.1% and 10% by weight of the total weight of the matrix composition.

According to certain embodiments, the matrix composition is homogeneous. In other embodiments, the matrix composition is in the form of a lipid-based matrix whose shape and boundaries are determined by the biocompatible polymer. In yet further embodiments, the matrix composition is in the form of an implant.

In certain particular embodiments, the present invention provides a matrix composition comprising: (a) biodegradable polyester; (b) a sterol; (c) a phosphatidylcholine having fatty acid moieties of at least 14 carbons; (d) a peptidic molecule and (e) PEG.

In other particular embodiments, the present invention provides a matrix composition comprising: (a) biodegradable polyester; (b) a sterol; (c) a phosphatidylcholine having a fatty acid moieties of at least 14 carbons; (d) a polar peptidic molecule and (e) PEG.

In yet other particular embodiments, the present invention provides a matrix composition comprising: (a) biodegradable polyester; (b) a sterol; (c) a phosphatidylcholine having a saturated fatty acid moieties of at least 14 carbons; (d) a polar peptidic molecule and (e) PEG.

In certain embodiments the matrix composition comprises at least 50% lipid by weight. In certain additional embodiments, the matrix composition further comprises a targeting moiety.

According to certain embodiments, the matrix composition is substantially free of water. The term “substantially free of water” refers to a composition containing less than 1% water by weight, less than 0.8% water by weight, less than 0.6% water by weight, less than 0.4% water by weight or less than 0.2% water by weight. Each possibility represents a separate embodiment of the present invention. In another embodiment, the term refers to the absence of amounts of water that affect the water-resistant properties of the matrix.

According to additional embodiments, the matrix composition is essentially free of water. “Essentially free” refers to composition comprising less than 0.1% water by weight, less than 0.08% water by weight, less than 0.06% water by weight, less than 0.04% water by weight or less than 0.02% water by weight. Each possibility represents a separate embodiment of the present invention. In another embodiment, the term refers to a composition comprising less than 0.01% water by weight.

According to further embodiments, each matrix composition is free of water. In another embodiment, the term refers to a composition not containing detectable amounts of water. Each possibility represents a separate embodiment of the present invention.

In certain embodiments, the matrix composition is capable of being degraded in vivo to vesicles into which some or all the mass of the released peptide, polypeptide or protein is integrated. In other embodiments, the matrix composition is capable of being degraded in vivo to form vesicles into which the active peptidic molecule and the targeting moiety are integrated.

According to an additional aspect the present invention provides a pharmaceutical composition comprising the matrix composition of the present invention and a pharmaceutically acceptable excipient.

According to certain embodiments, the matrix composition of the present invention is in the form of an implant, following removal of the organic solvents and water. In another embodiment, the implant is homogeneous. Each possibility represents a separate embodiment of the present invention.

According to certain embodiments, the process of creating an implant from a composition of the present invention comprises the steps of (a) creating a matrix composition according to a method of the present invention in the form of a bulk material; and (b) transferring the bulk material into a mold or solid receptacle of a desired shaped.

According to another aspect the present invention provides a method for producing a matrix composition for delivery and sustained and/or controlled release of a biologically active peptidic molecule comprising:

(a) mixing into a first solvent (i) a biocompatible polymer and (ii) a first lipid component comprising at least one lipid having a polar group;

(b) mixing the peptidic molecule into a second solvent to form a solution and adding polyethylene glycol into the solution;

(c) mixing the solution obtained in step (b) with a second lipid component comprising at least one phospholipid having fatty acid moieties of at least 14 carbons;

(d) mixing the solutions obtained in steps (a) and (c) to form a homogeneous mixture; and

(e) removing the solvents,

thereby producing a homogeneous polymer-phospholipids matrix comprising the peptidic molecule.

According to some embodiments, the first solvent is a volatile organic solvent. According to certain embodiments, the second solvent is selected from the group consisting of volatile organic solvent, a polar solvent and any mixtures thereof. According to typical embodiments, the polar solvent is water.

According to certain embodiments, step (c) optionally further comprises (i) removing the solvents by evaporation, freeze drying or centrifugation to form a sediment; and (ii) suspending the resulted sediment in the second volatile organic solvent.

The selection of the specific solvents is made according to the specific peptide, polypeptide or protein and the other substances used in a particular formulation and the intended use of the biologically active peptide, polypeptide or protein, and according to embodiments of the present invention described herein. The particular lipids forming the matrix of the present invention are selected according to the desired release rate of the peptide, polypeptide or protein and according to embodiments of the present invention described herein.

The solvents are typically removed by evaporation conducted at controlled temperature determined according to the properties of the solution obtained and the type of the biologically active peptidic molecule. Residues of the organic solvents and water are further removed using vacuum.

According to the present invention the use of different types of volatile organic solutions enable the formation of homogeneous water-resistant, lipid based matrix compositions. According to various embodiments the first and second solvents can be the same or different. According to some embodiments one solvent can be non-polar and the other water-miscible.

According to certain embodiments, the biodegradable polyester is selected from the group consisting of PLA, PGA and PLGA, chitosan and collagen. In other embodiments, the biodegradable polyester is any other suitable biodegradable polyester or polyamine known in the art.

In certain embodiments, the polymer in the mixture of step (a) is lipid saturated. In additional embodiments, the matrix composition is lipid saturated. Each possibility represents a separate embodiment of the present invention.

The matrix composition of the present invention can be used for coating fully or partially the surface of different substrates. According to certain embodiments, substrates to be coated include at least one material selected from the group consisting of carbon fibers, stainless steel, hydroxylapatite coated metals, synthetic polymers, rubbers, silicon, cobalt-chromium, titanium alloy, tantalum, ceramic and collagen or gelatin. In other embodiments substrates may include any medical devices and bone filler particles. Bone filler particles can be any one of allogeneic (i.e., from human sources), xenogeneic (i.e., from animal sources) and artificial bone particles. According to certain typical embodiments, the coating has a thickness of 1-200 μm; preferably between 5-100 μm. In other embodiments a treatment using the coated substrates and administration of the coated substrates will follow procedures known in the art for treatment and administration of similar uncoated substrates.

It is to be emphasized that the sustained release period using the compositions of the present invention can be programmed taking into account four major factors: (i) the weight ratio between the polymer and the lipid content, specifically the phospholipid having fatty acid moieties of at least 14 carbons, (ii) the biochemical and/or biophysical properties of the biopolymer and the lipids; (iii) the ratio between the different lipids used in a given composition. The incubation time of the peptide, polypeptide or protein with polyethylene glycol may also affect the sustained-release period.

Specifically, the degradation rate of the polymer and the fluidity of the lipid should be considered. For example, a PLGA (85:15) polymer will degrade slower than a PLGA (50:50) polymer. A phosphatidylcholine (14:0) is more fluid (less rigid and less ordered) at body temperature than a phosphatidylcholine (18:0). Thus, for example, the release rate of a peptidic molecule incorporated in a matrix composition comprising PLGA (85:15) and phosphatidylcholine (18:0) will be slower than that of the molecule incorporated in a matrix composed of PLGA (50:50) and phosphatidylcholine (14:0). Another aspect that will determine the release rate is the physical characteristics of the peptide, polypeptide or protein incorporated into the matrix. In addition, the release rate of a therapeutic peptidic molecule can further be controlled by the addition of other lipids into the formulation of the second lipid component. This can includes fatty acids of different length such as lauric acid (C12:0), membrane active sterols (such as cholesterol) or other phospholipids such as phosphatidylethanolamine. The incubation time of the peptide, polypeptide or protein with polyethylene glycol may also affects the release rate of the peptidic molecule from the matrix.

According to certain embodiments, at least 30% of the peptidic molecule is released from the matrix composition at zero-order kinetics. According to other embodiments, at least 50% of the peptidic molecule is released from the composition at zero-order kinetics.

These and other features and advantages of the present invention will become more readily understood and appreciated from the detailed description of the invention that follows.

### BRIEF DESCRIPTION OF THE DRAWINGS

FIG. 1 shows the release profile of NBD-labeled antimicrobial peptide from a matrix according to some embodiments of the invention.

### DETAILED DESCRIPTION OF THE INVENTION

The present invention provides compositions for extended and/or controlled release of peptidic molecules having therapeutic activity, comprising a lipid-based matrix with a biocompatible polymer. Particularly, the matrix compositions of the present invention are suitable for local release of the active molecule. The present invention also provides methods of producing the matrix compositions and methods for using the matrix compositions to provide controlled release of an active ingredient in the body of a subject in need thereof.

According to one aspect, the present invention provides a matrix composition comprising: (a) a pharmaceutically acceptable biocompatible polymer in association with a first lipid component comprising at least one lipid having a polar group; (b) a second lipid component comprising at least one phospholipid having fatty acid moieties of at least 14 carbons; (c) at least one peptidic molecule in association with polyethylene glycol (PEG), wherein the matrix composition is adapted for providing controlled release of the peptidic molecule. According to some embodiments, the peptidic molecule is polar.

According to certain embodiments, the biocompatible polymer is biodegradable. According to other embodiments, the biocompatible polymer is non-biodegradable. According to additional embodiments, the biocompatible polymer comprises a combination of biodegradable and non-biodegradable polymers, optionally as block co-polymer.

According to certain embodiments, the present invention provides a matrix composition comprising: (a) pharmaceutically acceptable biodegradable polyester; (b) a phospholipid having fatty acid moieties of at least 14 carbons: (c) a pharmaceutically active peptidic molecule; and (d) PEG.

The peptidic molecule can be any oligopeptide, polypeptide or protein having therapeutic effect. According to certain embodiments, the peptidic molecule is selected from an enzyme, a hormone, an antibody, an anti-microbial peptide, an anti-cancer peptide, an anti-cancer protein, an osteogenic factor a growth factor or a low oral bioavailability protein or peptide. Each possibility represents a separate embodiment of the invention. According to certain typical embodiments, the peptidic molecule is an anti-microbial peptide. According to other typical embodiments, the peptidic molecule is an enzyme.

According to some embodiments the lipid-saturated matrix composition comprises at least one cationic lipid. The term “cationic lipid” refers to any of a number of lipid species that carry a net positive charge at a selected pH, such as physiological pH. Such lipids include, but are not limited to, N,N-dioleyl-N,N-dimethylammonium chloride (“DODAC”); N-(2,3-dioleyloxy)propyl)-N,N,N-trimethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 3-(N—(N′,N′-dimethylaminoethane)carbamoyl)cholesterol (“DC-Chol”) and N-(1,2-dimyristyloxyprop-3-yl)-N,N-dimethyl-N-hydroxyethyl ammonium bromide (“DMRIE”). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (“DOPE”), from GIBCO/BRL, Grand Island, N.Y., USA); LIPOFECTAMINE® (commercially available cationic liposomes comprising N-(1-(2,3-dioleyloxy)propyl)-N-(2-(sperminecarboxamido)ethyl)N,N-dimethylammonium trifluoroacetate (“DOSPA”) and (“DOPE”), from GIBCO/BRL); and TRANSFECTAM® (commercially available cationic lipids comprising dioctadecylamidoglycyl carboxyspermine (“DOGS”) in ethanol from Promega Corp., Madison, Wis., USA). The following lipids are cationic and have a positive charge at below physiological pH: DODAP, DODMA, DMDMA and the like. Without wishing to be bound by any specific theory or mechanism of action, the cationic lipids of the matrix facilitate the internalization of the matrix of the invention, comprising peptidic molecule, into cells or tissues. According to certain embodiments, the cells and/or tissues form part of the human body.

According to other embodiments the biodegradable polymer comprises cationic polymers, such as cationized guar gum, diallyl quaternary ammonium salt/acrylamide copolymers, quaternized polyvinylpyrrolidone and derivatives thereof, and various polyquaternium-compounds.

According to certain embodiments, the phospholipid of the second lipid component is a phosphatidylcholine having fatty acid moieties of at least 14 carbons. In another embodiment, the second lipid component further comprises a phosphatidylethanolamine having fatty acid moieties of at least 14 carbons. In another embodiment, the second lipid component further comprises sterol, particularly cholesterol.

In certain embodiments, the matrix composition is lipid saturated. “Lipid saturated” as used herein, refers to saturation of the polymer of the matrix composition with lipids including phospholipids, in combination with any peptidic molecule and optionally a targeting moiety present in the matrix, and any other lipids that may be present. The matrix composition is saturated by whatever lipids are present. Lipid-saturated matrices of the present invention exhibit the additional advantage of not requiring a synthetic emulsifier or surfactant such as polyvinyl alcohol; thus, compositions of the present invention are typically substantially free of polyvinyl alcohol. Methods for determining the polymer:lipid ratio to attain lipid saturation and methods of determining the degree of lipid saturation of a matrix are known in the art.

In other embodiments, the matrix composition is homogeneous. In yet additional embodiments, the matrix composition is in the form of a lipid-saturated matrix whose shape and boundaries are determined by the biocompatible polymer. According to certain embodiments, the matrix composition is in the form of an implant.

In certain particular embodiments, the present invention provides a matrix composition comprising: (a) biodegradable polyester; (b) a sterol; (c) a phosphatidylcholine having fatty acid moieties of at least 14 carbons; (d) at least one peptidic molecule having therapeutic effect, and (c) PEG. In other typical embodiments, the matrix composition is lipid saturated. In other typical embodiments, the peptidic molecule is polar. In yet other typical embodiments, the phosphatidylcholine is having saturated fatty acid moieties of at least 14 carbons.

According to certain embodiments, the biodegradable polyester is associated with the sterol via non-covalent bonds.

As provided herein, the matrix of the present invention is capable of being molded into three-dimensional configurations of varying thickness and shape. Accordingly, the matrix formed can be produced to assume a specific shape including a sphere, cube, rod, tube, sheet, or into strings. In the case of employing freeze-drying steps during the preparation of the matrix, the shape is determined by the shape of a mold or support which may be made of any inert material and may be in contact with the matrix on all sides, as for a sphere or cube, or on a limited number of sides as for a sheet. The matrix may be shaped in the form of body cavities as required for implant design. Removing portions of the matrix with scissors, a scalpel, a laser beam or any other cutting instrument can create any refinements required in the three-dimensional structure. Each possibility represents a separate embodiment of the present invention.

According to additional embodiments, the matrix composition of the present invention provides a coating of bone graft material. According to certain embodiment, the bone graft material is selected from the group consisting of an allograft, an alloplast, and xenograft. According to further embodiments the matrix of the present invention can be combined with a collagen or collagen matrix protein. According to additional embodiments, the matrix can be sued for coating hydroxylapatite coated metals, synthetic polymers, rubbers and silicon substrates. According to some embodiments, the coating has a thickness of less than 200 μm; alternatively, less than 150 μm; alternatively, less than 100 μm; alternatively, less than 90 μm; alternatively, less than 80 μm; alternatively, less than 70 μm; alternatively, less than 60 μm; alternatively, less than 50 μm.

### Lipids

“Phosphatidylcholine” refers to a phosphoglyceride having a phosphorylcholine head group. Phosphatidylcholine compounds, in another embodiment, have the following structure:

[chemical structure]

The R₁ and R₂ moieties are fatty acids, typically naturally occurring fatty acids or derivatives of naturally occurring fatty acids. In some embodiments, the fatty acid moieties are saturated fatty acid moieties. In some embodiments, the fatty acid moieties are unsaturated fatty acid moieties. In some embodiments, at least one fatty acid moiety is saturated. In some currently preferred embodiments, both fatty acid moieties are saturated. “Saturated”, refers to the absence of a double bond in the hydrocarbon chain. In another embodiment, the fatty acid moieties have at least 14 carbon atoms. In another embodiment, the fatty acid moieties have 16 carbon atoms. In another embodiment, the fatty acid moieties have 18 carbon atoms. In another embodiment, the fatty acid moieties have 16-18 carbon atoms. In another embodiment, the fatty acid moieties are chosen such that the gel-to-liquid-crystal transition temperature of the resulting matrix is at least 40° C. In another embodiment, the fatty acid moieties are both palmitoyl. In another embodiment, the fatty acid moieties are both stearoyl. In another embodiment, the fatty acid moieties are both arachidoyl. In another embodiment, the fatty acid moieties are palmitoyl and stearoyl. In another embodiment, the fatty acid moieties are palmitoyl and arachidoyl. In another embodiment, the fatty acid moieties are arachidoyl and stearoyl. In another embodiment, the fatty acid moieties are both myristoyl. Each possibility represents a separate embodiment of the present invention.

In another embodiment, the phosphatidylcholine is a naturally-occurring phosphatidylcholine. In another embodiment, the phosphatidylcholine is a synthetic phosphatidylcholine. In another embodiment, the phosphatidylcholine contains a naturally-occurring distribution of isotopes. In another embodiment, the phosphatidylcholine is a deuterated phosphatidylcholine. Typically, the phosphatidylcholine is a symmetric phosphatidylcholine (i.e. a phosphatidylcholine wherein the two fatty acid moieties are identical). In another embodiment, the phosphatidylcholine is an asymmetric phosphatidylcholine.

Non-limiting examples of phosphatidylcholines are 1,2-distearoyl-sn-glycero-3-phosphocholine (DSPC), Dipalmitoyl-phosphatidylcholine (DPPC), Dimyristoyl-phosphatidylcholine (DMPC), dioleoyl-phosphatidylcholine (DOPC), 1-palmitoyl-2-oleoyl-phosphatidylcholine, and phosphatidylcholines modified with any of the fatty acid moieties enumerated hereinabove. In certain embodiments, the phosphatidylcholine is selected from the group consisting of DSPC, DPPC and DMPC. In another embodiment, the phosphatidylcholine is any other phosphatidylcholine known in the art. Each phosphatidylcholine represents a separate embodiment of the present invention.

Non-limiting examples of deuterated phosphatidylcholines are deuterated 1,2-distearoyl-sn-glycero-3-phosphocholine (deuterated DSPC), deuterated dioleoyl-phosphatidylcholine (deuterated DOPC), and deuterated 1-palmitoyl-2-oleoyl-phosphatidyl choline. In another embodiment, the phosphatidylcholine is selected from the group consisting of deuterated DSPC, deuterated DOPC, and deuterated 1-palmitoyl-2-oleoyl-phosphatidylcholine. In another embodiment, the phosphatidylcholine is any other deuterated phosphatidylcholine known in the art.

In certain embodiments, the phosphatidylcholine(s) (PC) compose at least 30% of the total lipid content of the matrix composition. In other embodiments, PC(s) compose at least 35% of the total lipid content, alternatively at least 40% of the total lipid content, yet alternatively at least 45%, at least 50%, least 55%, least 60%, at least 65%, at least 70%, at least 75%, at least 80%, at least 85%, at least 90% or at least 95% of the total lipid content. In another embodiment, PC(s) compose over 95% of the total lipid content. Each possibility represents a separate embodiment of the present invention.

“Phosphatidylethanolamine” refers to a phosphoglyceride having a phosphoryl ethanolamine head group. Phosphatidylethanolamine compounds, in another embodiment, have the following structure:

[chemical structure]

The R₁ and R₂ moieties are fatty acids, typically naturally occurring fatty acids or derivatives of naturally occurring fatty acids. In another embodiment, the fatty acid moieties are saturated fatty acid moieties. “Saturated” in another embodiment, refers to the absence of a double bond in the hydrocarbon chain. In another embodiment, the fatty acid moieties have at least 14 carbon atoms. In another embodiment, the fatty acid moieties have at least 16 carbon atoms. In another embodiment, the fatty acid moieties have 14 carbon atoms. In another embodiment, the fatty acid moieties have 16 carbon atoms. In another embodiment, the fatty acid moieties have 18 carbon atoms. In another embodiment, the fatty acid moieties have 14-18 carbon atoms. In another embodiment, the fatty acid moieties have 14-16 carbon atoms. In another embodiment, the fatty acid moieties have 16-18 carbon atoms. In another embodiment, the fatty acid moieties are chosen such that the gel-to-liquid-crystal transition temperature of the resulting matrix is at least 40° C. In another embodiment, the fatty acid moieties are both myristoyl. In another embodiment, the fatty acid moieties are both palmitoyl. In another embodiment, the fatty acid moieties are both stearoyl. In another embodiment, the fatty acid moieties are both arachidoyl. In another embodiment, the fatty acid moieties are myristoyl and stearoyl. In another embodiment, the fatty acid moieties are myristoyl and arachidoyl. In another embodiment, the fatty acid moieties are myristoyl and palmitoyl. In another embodiment, the fatty acid moieties are palmitoyl and stearoyl. In another embodiment, the fatty acid moieties are palmitoyl and arachidoyl. In another embodiment, the fatty acid moieties are arachidoyl and stearoyl. Each possibility represents a separate embodiment of the present invention.

In another embodiment, the phosphatidylethanolamine is a naturally-occurring phosphatidylethanolamine. In another embodiment, the phosphatidylethanolamine is a synthetic phosphatidylethanolamine. In another embodiment, the phosphatidylethanolamine is a deuterated phosphatidylethanolamine. In another embodiment, the phosphatidylethanolamine contains a naturally-occurring distribution of isotopes. Typically the phosphatidylethanolamine is a symmetric phosphatidylethanolamine. In another embodiment, the phosphatidylethanolamine is an asymmetric phosphatidylethanolamine.

Non-limiting examples of phosphatidylethanolamines are dimethyl dimyristoyl phosphatidylethanolamine (DMPE) and dipalmitoyl-phosphatidylethanolamine (DPPE), and phosphatidylethanolamines modified with any of the fatty acid moieties enumerated hereinabove. In another embodiment, the phosphatidylethanolamine is selected from the group consisting of DMPE and DPPE.

Non-limiting examples of deuterated phosphatidylethanolamines are deuterated DMPE and deuterated DPPE. In another embodiment, the phosphatidylethanolamine is selected from the group consisting of deuterated DMPE and deuterated DPPE. In another embodiment, the phosphatidylethanolamine is any other deuterated phosphatidylethanolamine known in the art.

In another embodiment, the phosphatidylethanolamine is any other phosphatidylethanolamine known in the art. Each phosphatidylethanolamine represents a separate embodiment of the present invention.

“Sterol” in one embodiment refers to a steroid with a hydroxyl group at the 3-position of the A-ring. In another embodiment, the term refers to a steroid having the following structure:

[chemical structure]

In another embodiment, the sterol of methods and compositions of the present invention is a zoosterol. In another embodiment, the sterol is cholesterol:

[chemical structure]

In another embodiment, the sterol is any other zoosterol known in the art. In another embodiment, the moles of sterol are up to 40% of the moles of total lipids present. In another embodiment, the sterol is incorporated into the matrix composition. Each possibility represents a separate embodiment of the present invention.

In another embodiment, the cholesterol is present in an amount of 10-60 percentage of the total weight of lipid content of the matrix composition. In another embodiment, the weight percentage is 20-50%. In another embodiment, the weight percentage is 10-40%. In another embodiment, the weight percentage is 30-50%. In another embodiment, the weight percentage is 20-60%. In another embodiment, the weight percentage is 25-55%. In another embodiment, the weight percentage is 35-55%. In another embodiment, the weight percentage is 30-60%. In another embodiment, the weight percentage is 30-55%. In another embodiment, the weight percentage is 20-50%. In another embodiment, the weight percentage is 25-55%. Each possibility represents a separate embodiment of the present invention.

In another embodiment, a composition of the present invention further comprises a lipid other than phosphatidylcholine, phosphatidylethanolamine, or a sterol. According to certain embodiments, the additional lipid is a phosphoglyceride. According to other embodiments, the additional lipid is selected from the group consisting of a phosphatidylserine, a phosphatidylglycerol, and a phosphatidylinositol. In yet additional embodiments, the additional lipid is selected from the group consisting of a phosphatidylserine, a phosphatidylglycerol, a phosphatidylinositol, and a sphingomyelin. According to yet further embodiments, a combination of any 2 or more of the above additional lipids is present within the matrix of the invention. According to certain embodiments, the polymer, phosphatidylcholine, phosphatidylethanolamine, sterol, and additional lipid(s) are all incorporated into the matrix composition. Each possibility represents a separate embodiment of the present invention.

According to yet additional embodiments, a composition of the present invention further comprises a phosphatidylserine. As used herein, “phosphatidylserine” refers to a phosphoglyceride having a phosphorylserine head group. Phosphatidylserine compounds, in another embodiment, have the following structure:

[chemical structure]

The R₁ and R₂ moieties are fatty acids, typically naturally occurring fatty acids or derivatives of naturally occurring fatty acids. In another embodiment, the fatty acid moieties are saturated fatty acid moieties. In another embodiment, the fatty acid moieties have at least 14 carbon atoms. In another embodiment, the fatty acid moieties have at least 16 carbon atoms. In another embodiment, the fatty acid moieties are chosen such that the gel-to-liquid-crystal transition temperature of the resulting matrix is at least 40° C. In another embodiment, the fatty acid moieties are both myristoyl. In another embodiment, the fatty acid moieties are both palmitoyl. In another embodiment, the fatty acid moieties are both stearoyl. In another embodiment, the fatty acid moieties are both arachidoyl. In another embodiment, the fatty acid moieties are myristoyl and stearoyl. In another embodiment, the fatty acid moieties are a combination of two of the above fatty acid moieties.

In other embodiments, the phosphatidylserine is a naturally-occurring phosphatidyl serine. In another embodiment, the phosphatidylserine is a synthetic phosphatidyl serine. In another embodiment, the phosphatidylserine is a deuterated phosphatidyl serine. In another embodiment, the phosphatidylserine contains a naturally-occurring distribution of isotopes. In another embodiment, the phosphatidylserine is a symmetric phosphatidylserine. In another embodiment, the phosphatidylserine is an asymmetric phosphatidylserine.

Non-limiting examples of phosphatidylserines are phosphatidylserines modified with any of the fatty acid moieties enumerated hereinabove. In another embodiment, the phosphatidylserine is any other phosphatidylserine known in the art. Each phosphatidylserine represents a separate embodiment of the present invention.

In other embodiments, a composition of the present invention further comprises a phosphatidylglycerol. “Phosphatidylglycerol” as used herein refers to a phosphoglyceride having a phosphoryl glycerol head group. Phosphatidylglycerol compounds, in another embodiment, have the following structure:

[chemical structure]

The 2 bonds to the left are connected to fatty acids, typically naturally occurring fatty acids or derivatives of naturally occurring fatty acids. In another embodiment, the phosphatidylglycerol is a naturally-occurring phosphatidylglycerol. In another embodiment, the phosphatidylglycerol is a synthetic phosphatidyl glycerol. In another embodiment, the phosphatidylglycerol is a deuterated phosphatidylglycerol. In another embodiment, the phosphatidylglycerol contains a naturally-occurring distribution of isotopes. In another embodiment, the phosphatidylglycerol is a symmetric phosphatidylglycerol. In another embodiment, the phosphatidylglycerol is an asymmetric phosphatidylglycerol. In another embodiment, the term includes diphosphatidylglycerol compounds having the following structure:

[chemical structure]

The R₁, R₂, R₃ and R₄ moieties are fatty acids, typically naturally occurring fatty acids or derivatives of naturally occurring fatty acids. In another embodiment, the fatty acid moieties are saturated fatty acid moieties. In another embodiment, the fatty acid moieties have at least 14 carbon atoms. In another embodiment, the fatty acid moieties have at least 16 carbon atoms. In another embodiment, the fatty acid moieties are chosen such that the gel-to-liquid-crystal transition temperature of the resulting matrix is at least 40° C. In another embodiment, the fatty acid moieties are both myristoyl. In another embodiment, the fatty acid moieties are both palmitoyl. In another embodiment, the fatty acid moieties are both stearoyl. In another embodiment, the fatty acid moieties are both arachidoyl. In another embodiment, the fatty acid moieties are myristoyl and stearoyl. In another embodiment, the fatty acid moieties are a combination of two of the above fatty acid moieties.

Non-limiting examples of phosphatidylglycerols are phosphatidylglycerols modified with any of the fatty acid moieties enumerated hereinabove. In another embodiment, the phosphatidylglycerol is any other phosphatidylglycerol known in the art. Each phosphatidylglycerol represents a separate embodiment of the present invention.

In yet additional embodiments, a composition of the present invention further comprises a phosphatidylinositol. As used herein, “phosphatidyl inositol” refers to a phosphoglyceride having a phosphorylinositol head group. Phosphatidylinositol compounds, in another embodiment, have the following structure:

[chemical structure]

The R and R′ moieties are fatty acids, typically naturally occurring fatty acids or derivatives of naturally occurring fatty acids. In another embodiment, the fatty acid moieties are saturated fatty acid moieties. In another embodiment, the fatty acid moieties have at least 14 carbon atoms. In another embodiment, the fatty acid moieties have at least 16 carbon atoms. In another embodiment, the fatty acid moieties are chosen such that the gel-to-liquid-crystal transition temperature of the resulting matrix is at least 40° C. In another embodiment, the fatty acid moieties are both myristoyl. In another embodiment, the fatty acid moieties are both palmitoyl. In another embodiment, the fatty acid moieties are both stearoyl. In another embodiment, the fatty acid moieties are both arachidoyl. In another embodiment, the fatty acid moieties are myristoyl and stearoyl. In another embodiment, the fatty acid moieties are a combination of two of the above fatty acid moieties.

In another embodiment, the phosphatidyl inositol is a naturally-occurring phosphatidylinositol. In another embodiment, the phosphatidylinositol is a synthetic phosphatidylinositol. In another embodiment, the phosphatidylinositol is a deuterated phosphatidylinositol. In another embodiment, the phosphatidylinositol contains a naturally-occurring distribution of isotopes. In another embodiment, the phosphatidylinositol is a symmetric phosphatidylinositol. In another embodiment, the phosphatidylinositol is an asymmetric phosphatidylinositol.

Non-limiting examples of phosphatidylinositols are phosphatidylinositols modified with any of the fatty acid moieties enumerated hereinabove. In another embodiment, the phosphatidylinositol is any other phosphatidylinositol known in the art. Each phosphatidylinositol represents a separate embodiment of the present invention.

In further embodiments, a composition of the present invention further comprises a sphingolipid. In certain embodiments, the sphingolipid is ceramide. In yet other embodiments, the sphingolipid is a sphingomyelin. “Sphingomyelin” refers to a sphingosine-derived phospholipid. Sphingomyelin compounds, in another embodiment, have the following structure:

[chemical structure]

The R moiety is a fatty acid, typically a naturally occurring fatty acid or a derivative of a naturally occurring fatty acid. In another embodiment, the sphingomyelin is a naturally-occurring sphingomyelin. In another embodiment, the sphingomyelin is a synthetic sphingomyelin. In another embodiment, the sphingomyelin is a deuterated sphingomyelin. In another embodiment, the sphingomyelin contains a naturally-occurring distribution of isotopes.

In another embodiment, the fatty acid moiety of a sphingomyelin of methods and compositions of the present invention has at least 14 carbon atoms. In another embodiment, the fatty acid moiety has at least 16 carbon atoms. In another embodiment, the fatty acid moiety is chosen such that the gel-to-liquid-crystal transition temperature of the resulting matrix is at least 40° C.

Non-limiting examples of sphingomyelins are sphingomyelins modified with any of the fatty acid moieties enumerated hereinabove. In another embodiment, the sphingomyelin is any other sphingomyelin known in the art. Each sphingomyelin represents a separate embodiment of the present invention.

“Ceramide” refers to a compound having the structure:

[chemical structure]

The 2 bonds to the left are connected to fatty acids, typically naturally occurring fatty acids or derivatives of naturally occurring fatty acids. In another embodiment, the fatty acids are longer-chain (to C₂₄ or greater). In another embodiment, the fatty acids are saturated fatty acids. In another embodiment, the fatty acids are monoenoic fatty acids. In another embodiment, the fatty acids are n-9 monoenoic fatty acids. In another embodiment, the fatty acids contain a hydroxyl group in position 2. In another embodiment, the fatty acids are other suitable fatty acids known in the art. In another embodiment, the ceramide is a naturally-occurring ceramide. In another embodiment, the ceramide is a synthetic ceramide. In another embodiment, the ceramide is incorporated into the matrix composition. Each possibility represents a separate embodiment of the present invention.

Each sphingolipid represents a separate embodiment of the present invention.

In certain embodiments, a composition of the present invention further comprises a pegylated lipid. In another embodiment, the PEG moiety has a MW of 500-5000 daltons. In another embodiment, the PEG moiety has any other suitable MW. Non-limiting examples of suitable PEG-modified lipids include PEG moieties with a methoxy end group, e.g. PEG-modified phosphatidylethanolamine and phosphatidic acid (structures A and B), PEG-modified diacylglycerols and dialkylglycerols (structures C and D), PEG-modified dialkylamines (structure E) and PEG-modified 1,2-diacyloxypropan-3-amines (structure F) as depicted below. In another embodiment, the PEG moiety has any other end group used in the art. In another embodiment, the pegylated lipid is selected from the group consisting of a PEG-modified phosphatidylethanolamine, a PEG-modified phosphatidic acid, a PEG-modified diacylglycerol, a PEG-modified dialkylglycerol, a PEG-modified dialkylamine, and a PEG-modified 1,2-diacyloxypropan-3-amine. In another embodiment, the pegylated lipid is any other pegylated phospholipid known in the art. Each possibility represents a separate embodiment of the present invention.

[chemical structure]

According to certain embodiments, the pegylated lipid is present in an amount of about 50 mole percent of total lipids in the matrix composition. In other embodiments, the percentage is about 45 mole %, alternatively about 40 mole %, about 35 mole about 30 mole %, about 25 mole %, about 20 mole %, about 15 mole %, about 10 mole %, and about 5 mole % or less. Each possibility represents a separate embodiment of the present invention.

### Polymers

According to certain embodiments, the biocompatible polymer is biodegradable. According to certain currently typical embodiments, the biodegradable polymer is polyester.

According to certain embodiments, the biodegradable polyester employed according to the teachings of the present invention is PLA (polylactic acid). According to typical embodiments, “PLA” refers to poly(L-lactide), poly(D-lactide), and poly(DL-lactide). A representative structure of poly(DL-lactide) is depicted below:

[chemical structure]

In other embodiments, the polymer is PGA (polyglycolic acid). In yet additional embodiments, the polymer is PLGA (poly(lactic-co-glycolic acid). The PLA contained in the PLGA may be any PLA known in the art, e.g. either enantiomer or a racemic mixture. A representative structure of PLGA is depicted below:

[chemical structure]

According to certain embodiments, the PLGA comprises a 1:1 lactic acid/glycolic acid ratio. In another embodiment, the ratio is 60:40. In another embodiment, the ratio is 70:30. In another embodiment, the ratio is 80:20. In another embodiment, the ratio is 90:10. In another embodiment, the ratio is 95:5. In another embodiment, the ratio is another ratio appropriate for an extended in vivo release profile, as defined herein. In another embodiment, the ratio is 50:50. In certain typical embodiments, the ratio is 75:25. The PLGA may be either a random or block copolymer. The PLGA may be also a block copolymer with other polymers such as PEG. Each possibility represents a separate embodiment of the present invention.

In another embodiment, the biodegradable polyester is selected from the group consisting of a polycaprolactone, a polyhydroxyalkanoate, a polypropylenefumarate, a polyorthoester, a polyanhydride, and a polyalkylcyanoacrylate, provided that the polyester contains a hydrogen bond acceptor moiety. In another embodiment, the biodegradable polyester is a block copolymer containing a combination of any two monomers selected from the group consisting of a PLA, PGA, a PLGA, polycaprolactone, a polyhydroxyalkanoate, a polypropylenefumarate, a polyorthoester, a polyanhydride, and a polyalkylcyanoacrylate. In another embodiment, the biodegradable polyester is a random copolymer containing a combination of any two of the monomers listed above. Each possibility represents a separate embodiment of the present invention.

The molecular weight (MW) of a biodegradable polyester according to the teachings of the present invention is, in another embodiment, between about 10-150 KDa. In another embodiment, the MW is between about 20-150 KDa. In another embodiment, the MW is between about 10-140 KDa. In another embodiment, the MW is between about 20-130 KDa. In another embodiment, the MW is between about 30-120 KDa. In another embodiment, the MW is between about 45-120 KDa. In another typical embodiment, the MW is between about 60-110 KDa. In another embodiment, a mixture of PLGA polymers of different MW is utilized. In another embodiment, the different polymers both have a MW in one of the above ranges. Each possibility represents a separate embodiment of the present invention.

In another embodiment, the biodegradable polymer is selected from the group of polyamines consisting of peptides containing one or more types of amino acids, with at least 10 amino acids.

“Biodegradable,” as used herein, refers to a substance capable of being decomposed by natural biological processes at physiological pH. “Physiological pH” refers to the pH of body tissue, typically between 6-8. “Physiological pH” does not refer to the highly acidic pH of gastric juices, which is typically between 1 and 3.

According to some embodiments, the biocompatible polymer is non-biodegradable polymer. According to certain embodiments, the non-biodegradable polymer may be selected from the group consisting of, yet not limited to, polyethylene glycol, polyethylene glycol (PEG) acrylate, polymethacrylates (e.g. PEG methacrylate, polymethylmethacrylate, polyethylmethacrylate, polybutylmethacrylate, poly-2-ethylhexylmethacrylate, polylaurylmethacrylate, polyhydroxylethyl methacrylate), poly-methylacrylate, 2-methacryloyloxyethylphosphorylcholine (MPC), polystyrene, derivatized polystyrene, polylysine, poly N-ethyl-4-vinyl-pyridinium bromide, silicone, ethylene-vinyl acetate copolymers, polyethylenes, polypropylenes, polytetrafluoroethylenes, polyurethanes, polyacrylates, polyvinyl acetate, ethylene vinyl acetate, polyethylene, polyvinyl chloride, polyvinyl fluoride, copolymers of polymers of ethylene-vinyl acetates and acyl substituted cellulose acetates, poly(vinyl imidazole), chlorosulphonate polyolefins, polyethylene oxide, and mixtures thereof.

### Peptidic Molecules

The term “peptidic molecule” as used herein is intended to include any structure comprised of one or more amino acids. Typically, the peptidic molecules are comprised of two or more amino acids, and are peptides, polypeptides or proteins. The matrices of the present invention can comprise peptidic molecule of a wide size range, including peptides, polypeptides and proteins. The amino acids forming all or a part of a peptidic molecule may be any of the twenty conventional, naturally occurring amino acids. According to certain embodiments, any one of the amino acids of the peptidic molecule may be replaced by a non-conventional amino acid. The replacement can be conservative or non conservative. Conservative replacements substitute the original amino acid with a non-conventional amino acid that resembles the original in one or more of its characteristic properties (e.g., charge, hydrophobicity, stearic bulk). The term “non-conventional amino acid” refers to amino acids other than conventional amino acids, and include, for example, isomers and modifications of the conventional amino acids, e.g., D-amino acids, non-protein amino acids, post-translationally modified amino acids, enzymatically modified amino acids, constructs or structures designed to mimic amino acids (e.g., α-α.-disubstituted amino acids, N-alkyl amino acids, lactic acid, β-alanine, naphthylalanine, 3-pyridylalanine, 4-hydroxyproline, O-phosphoserine, N-acetylserine, N-formylmethionine, 3-methylhistidine, 5-hydroxylysine, and nor-leucine), and other non-conventional amino acids, as described, for example, in U.S. Pat. No. 5,679,782. The peptidic molecules may also contain nonpeptidic backbone linkages, wherein the naturally occurring amide —CONH— linkage is replaced at one or more sites within the peptide backbone with a non-conventional linkage such as N-substituted amide, ester, thioamide, retropeptide (—NHCO—), retrothioamide (—NHCS), sulfonamido (—SO₂NH—), and/or peptoid (N-substituted glycine) linkages. Accordingly, the peptidic molecules according to the teachings of the present invention can include pseudopeptides and peptidomimetics. The peptides of this invention can be (a) naturally occurring, (b) produced by chemical synthesis, (c) produced by recombinant DNA technology, (d) produced by biochemical or enzymatic fragmentation of larger molecules, (e) produced by methods resulting from a combination of methods (a) through (d) listed above, or (f) produced by any other means for producing peptides as is known in the art.

It is to be explicitly understood that the term “peptidic molecule” encompasses a peptide, a polypeptide and a protein. According to currently preferred embodiments, the peptidic compound comprises at least one amino acid having a polar functional group.

A “peptide” refers to a polymer in which the monomers are amino acids linked together through amide bonds. “Peptides” are generally smaller than proteins, typically under 30-50 amino acids in total.

A “polypeptide” refers to a single polymer of amino acids, generally over 50 amino acids.

A “protein” as used herein refers to a polymer of amino acids typically over 50 amino acids. Derivatives, analogs and fragments of the peptides, polypeptides or proteins are encompassed in the present invention so long as they retain a therapeutic effect.

According to certain embodiments, the peptidic molecule has a therapeutic activity. According to certain embodiments, the peptidic molecule is selected from an enzyme, a hormone, an anti-microbial agent, an antibody an anti-cancer drug, an osteogenic factor, a growth or a low oral bioavailability protein or peptide. Each possibility represents a separate embodiment of the invention. According to certain typical embodiments, the peptidic molecule is an anti-microbial peptide.

According to some embodiments the peptidic molecule is an anti-inflammatory agent. Non limiting examples of a suitable peptidic anti-inflammatory agent may be selected from the group consisting of TNF, IL-1, IL-6, IL-8, IL-12, IL-15, IL-17, IL-18, GM-CSF, M-CSF, MCP-1, MIP-1, RANTES, ENA-78, OSM, FGF, and VEGF. A variety of anti-inflammatory agents contemplated for use in the present invention are described in US 2003/0176332, which is incorporated herein by reference.

Non limiting examples of anti-cancer agents that may be used according to some embodiments, may include, such therapies and molecules as, but not limited to: administration of an immunomodulatory molecule, such as, for example, a molecule selected from the group consisting of tumor antigens, antibodies, cytokines (such as, for example, interleukins (such as, for example, interleukin 2, interleukin 4, interleukin 12), interferons (such as, for example, interferon E1 interferon D, interferon alpha), tumor necrosis factor (TNF), granulocyte macrophage colony stimulating factor (GM-CSF), macrophage colony stimulating factor (M-CSF), and granulocyte colony stimulating factor (G-CSF)), tumor suppressor genes, chemokines, complement components and complement component receptors.

In another embodiment, the active agent of methods and compositions of the present invention is a compound which induces or stimulates the formation of bone. In another embodiment the active agent is osteoinductive factor (also referred to as osteogenic factor). In another embodiment, the osteogenic factor refers to any peptide, polypeptide, protein which induces or stimulates the formation of bone. In another embodiment, the osteogenic factor induces differentiation of bone repair cells into bone cells, such as osteoblasts or osteocytes. According to some embodiments the osteoinductive factors are the recombinant human bone morphogenetic proteins (rhBMPs). Most preferably, the bone morphogenetic protein is a rhBMP-2, rhBMP-7 or heterodimers thereof. However, any bone morphogenetic protein is contemplated, including bone morphogenetic proteins designated as BMP-1 through BMP-13. BMPs are available from Genetics Institute, Inc., Cambridge, Mass. and may also be prepared by one skilled in the art, as described for example in U.S. Pat. No. 5,187,076, U.S. Pat. No. 5,366,875, U.S. Pat. No. 4,877,864, U.S. Pat. No. 5,108,922, U.S. Pat. No. 5,116,738, U.S. Pat. No. 5,013,649, U.S. Pat. No. 5,106,748. The osteoinductive factors that may be included in the matrix compositions according to embodiments of the invention may be obtained by any of the above know in the art methods or isolated from bone. Methods for isolating bone morphogenetic protein from bone are described in U.S. Pat. No. 4,294,753.

The growth factors may include but are not limited to bone morphogenic proteins, which have been shown to be excellent at growing bone, for example, BMP-1, BMP-2, rhBMP-2, BMP-3, BMP-4, rhBMP-4, BMP-5, BMP-6, rhBMP-6, BMP-7 [OP-1], rhBMP-7, BMP-8, BMP-9, BMP-10, BMP-11, BMP-12, BMP-13, BMP-14, BMP-15, BMP-16, BMP-17, BMP-18, GDF-5, and rhGDF-5, as disclosed, for example, in the U.S. Pat. No. 7,833,270.

Additionally, suitable growth factors include, without limitation, Cartilage Derived Morphogenic Proteins, LIM mineralization protein, platelet derived growth factor (PDGF), vascular endothelial growth factor (VEGF), transforming growth factor β (TGF-β), insulin-related growth factor-I (IGF-I), insulin-related growth factor-II (IGF-II), fibroblast growth factor (FGF), and beta-2-microglobulin (BDGF II), as disclosed in U.S. Pat. No. 7,833,270.

### Polyethylene Glycol

The present invention is based in part on the unexpected discovery that incubation of a peptidic molecule dissolved in adequate solvent with polyethylene glycol (PEG) enhances the capture of the peptidic molecule within the lipid-based matrix and affects the release rate of the molecule from the matrix under suitable conditions. The solvent may be an organic volatile solvent, a water miscible solvent or water, depending on the type of the peptidic molecule. As commonly used in the art, poly(ethylene) glycol generally refers to the linear form of poly(ethylene glycol) since these are the most common, commercially available PEG. Linear PEG can be represented by the formula OH—(CH₂CH₂O)ₙ—OH (diol) or mPEG, CH₃O—(CH₂CH₂O)ₙOH, wherein n is the average number of repeating ethylene oxide groups. These PEG compounds are commercially available from, e.g., Sigma-Aldrich in a variety of molecular weights ranging from 1000 to 300,000. Linear PEGs are available as monofunctional or bifunctional forms. PEG's may contain functional reactive groups at either end of the chain and can be homobifunctional (two identical reactive groups) or heterobifunctional (two different reactive groups). For example, heterobifunctional PEG of the formula _{NH2}—(CH₂CH₂O)ₙCOOH are commercially available and are useful for forming PEG derivatives. There are many grades of PEG compounds that are represented by theirs average molecular weight. Pharmaceutical grade PEG is typically in a molecular range of up to 8,000. According to certain typical embodiments, the PEG used according to the teachings of the present invention has a molecular weight of up to 5,000, typically about 2,000-5000.

According to some embodiments, PEG is present in an amount of between 0.1% and 10% by weight of the total weight of the matrix composition. According to certain embodiments, PEG is present in an amount of between 0.1% and 5% by weight of the total weight of the matrix composition. According to certain embodiments, PEG is present in an amount of between 0.1% and 2% by weight of the total weight of the matrix composition. According to some embodiments the weight ratio of the peptidic molecule and PEG is between 20:1 and 1:5. According to certain embodiments the weight ratio of the peptidic molecule and PEG is between 20:1 and 1:1. According to certain typical embodiments the weight ratio of the peptidic molecule and PEG is between 10:1 and 1:1.

### Additional Components

The matrix composition of the present invention optionally further comprises a free fatty acid. In certain embodiments, the free fatty acid is an omega-6 fatty acid. In other embodiments, the free fatty acid is an omega-9 fatty acid. In another embodiment, the free fatty acid is selected from the group consisting of omega-6 and omega-9 fatty acids. In further embodiments, the free fatty acid has 14 or more carbon atoms. In another embodiment, the free fatty acid has 16 or more carbon atoms. In another embodiment, the free fatty acid has 16 carbon atoms. In another embodiment, the free fatty acid has 18 carbon atoms. In another embodiment, the free fatty acid has 16-22 carbon atoms. In another embodiment, the free fatty acid has 16-20 carbon atoms. In another embodiment, the free fatty acid has 16-18 carbon atoms. In another embodiment, the free fatty acid has 18-22 carbon atoms. In another embodiment, the free fatty acid has 18-20 carbon atoms. In another embodiment, the free fatty acid is linoleic acid. In another embodiment, the free fatty acid is linolenic acid. In another embodiment, the free fatty acid is oleic acid. In another embodiment, the free fatty acid is selected from the group consisting of linoleic acid, linolenic acid, and oleic acid. In another embodiment, the free fatty acid is another appropriate free fatty acid known in the art. In another embodiment, the free fatty acid adds flexibility to the matrix composition. In another embodiment, the free fatty acid slows the release rate, including the in vivo release rate. In another embodiment, the free fatty acid improves the consistency of the controlled release, particularly in vivo. In another embodiment, the free fatty acid is saturated. In another embodiment, incorporation of a saturated fatty acid having at least 14 carbon atoms increases the gel-fluid transition temperature of the resulting matrix composition.

In another embodiment, the free fatty acid is incorporated into the matrix composition.

In another embodiment, the free fatty acid is deuterated. In another embodiment, deuteration of the lipid acyl chains lowers the gel-fluid transition temperature.

Each type of fatty acid represents a separate embodiment of the present invention.

According to certain embodiments, a matrix composition of the present invention further comprises a tocopherol. The tocopherol is, in another embodiment, E307 (α-tocopherol). In another embodiment, the tocopherol is γ-tocopherol. In another embodiment, the tocopherol is E308 (γ-tocopherol). In another embodiment, the tocopherol is E309 (6-tocopherol). In another embodiment, the tocopherol is selected from the group consisting of α-tocopherol, β-tocopherol, γ-tocopherol, and δ-tocopherol. In another embodiment, the tocopherol is incorporated into the matrix composition. Each possibility represents a separate embodiment of the present invention.

The matrix composition of the present invention optionally further comprises physiologically acceptable buffer salts, which are well known in the art. Non-limiting examples of physiologically acceptable buffer salts are phosphate buffers. A typical example of a phosphate buffer is 40 parts NaCl, 1 part KCl, 7 parts Na₂HPO₄.2H₂O and 1 part KH₂PO₄. In another embodiment, the buffer salt is any other physiologically acceptable buffer salt known in the art. Each possibility represents a separate embodiment of the present invention.

### Release Rates and General Characteristics of the Matrix Compositions

The release time of 90% of the active ingredient for matrix compositions of the present invention under suitable conditions is preferably between 4 days and 6 months. According to certain embodiments, the release time is between 1 week and 6 months, between 1 week and 5 months, between 1 week and 5 months, between 1 week and 4 months, between 1 week and 3 months, between 1 week and 2 months, or between 1 week and 1 month. Each possibility represents a separate embodiment of the present invention.

The sustained release period using the compositions of the present invention can be programmed taking into account three major factors: (i) the weight ratio between the polymer and the lipid content, specifically the phospholipid having fatty acid moieties of at least 14 carbons, (ii) the biochemical and/or biophysical properties of the biopolymers and the lipids used; and (iii) the ratio between the different lipids used in a given composition. The incubation time of the peptide, polypeptide or protein with polyethylene glycol may also affect the release rate.

The ratio of total lipids to the polymer in order to achieve lipid saturation can be determined by a number of methods, as described herein. According to certain embodiments, the lipid:polymer weight ratio of a composition of the present invention is between 1:1 and 9:1 inclusive. In another embodiment, the ratio is between 1.5:1 and 9:1 inclusive. In another embodiment, the ratio is between 2:1 and 9:1 inclusive. In another embodiment, the ratio is between 3:1 and 9:1 inclusive. In another embodiment, the ratio is between 4:1 and 9:1 inclusive. In another embodiment, the ratio is between 5:1 and 9:1 inclusive. In another embodiment, the ratio is between 6:1 and 9:1 inclusive. In another embodiment, the ratio is between 7:1 and 9:1 inclusive. In another embodiment, the ratio is between 8:1 and 9:1 inclusive. In another embodiment, the ratio is between 1.5:1 and 5:1 inclusive. Each possibility represents a separate embodiment of the present invention.

In another embodiment for purposes of illustration, in the case wherein the polymer is predominantly 40 KDa PLGA (poly(lactic-co-glycolic acid, 1:1 ratio)), the molar ratio of total lipids to 40 KDa PLGA is typically in the range of 20-100 inclusive. In another embodiment, the molar ratio of total lipids to 40 KDa PLGA is between 20-200 inclusive. In another embodiment, the molar ratio is between 10-100 inclusive. In another embodiment, the molar ratio is between 10-200 inclusive. In another embodiment, the molar ratio is between 10-50 inclusive. In another embodiment, the molar ratio is between 20-50 inclusive. Each possibility represents a separate embodiment of the present invention.

### Implants and Other Pharmaceutical Compositions

The matrix composition of the present invention can be molded to the form of an implant, following removal of the organic solvents and water. The removal of the solvents is typically performed by evaporation under a specific temperature which does not cause denaturation of the peptidic molecule between room temperature and 60° C., followed by vacuum. According to certain typically embodiments the evaporation temperature is blow 50° C. Each possibility represents a separate embodiment of the present invention.

In another embodiment, the implant is homogeneous. In another embodiment, the implant is manufactured by a process comprising the step of freeze-drying the material in a mold. Each possibility represents a separate embodiment of the present invention.

According to additional embodiments, the present invention provides an implant comprising a matrix composition comprising a peptidic molecule according to the teachings of the present invention.

The present invention further provides a process of creating an implant from a composition of the present invention comprising the steps of (a) creating a matrix composition according to the method of the present invention in the form of a bulk material; (b) transferring the bulk material into a mold or solid receptacle of a desired shaped; (c) freezing the bulk material; and (d) lyophilizing the bulk material.

In additional embodiments, the present invention provides a pharmaceutical composition comprising a matrix composition of the present invention. According to certain embodiments, the pharmaceutical composition further comprises additional pharmaceutically acceptable excipients. In additional embodiments, the pharmaceutical composition is in a parenterally injectable form. In other embodiments, the pharmaceutical composition is in an infusible form. In yet additional embodiments, the excipient is compatible for injection. In further embodiments, the excipient is compatible for infusion. Each possibility represents a separate embodiment of the present invention.

Use of the matrix composition of the present invention for the production of micro-vesicles, ranging from 100 nm to 50 mm is also within the scope of the present invention.

According to certain embodiments, the matrix composition of the present invention is in the form of microspheres, following removal of the organic solvents and water. In other embodiment, the microspheres are homogeneous. According to certain embodiments, the microspheres are manufactured by a process comprising the step of spray-drying. Each possibility represents a separate embodiment of the present invention.

In another embodiment, the present invention provides microspheres made of a matrix composition of the present invention. In another embodiment, the present invention provides a pharmaceutical composition comprising microspheres of the present invention and a pharmaceutically acceptable excipient. Each possibility represents a separate embodiment of the present invention.

In another embodiment, the particle size of microspheres of the present invention is approximately 500-2000 nm. In another embodiment, the particle size is about 400-2500 nm. In another embodiment, the particle size is about 600-1900 nm. In another embodiment, the particle size is about 700-1800 nm. In another embodiment, the particle size is about 500-1800 nm. In another embodiment, the particle size is about 500-1600 nm. In another embodiment, the particle size is about 600-2000 nm. In another embodiment, the particle size is about 700-2000 nm. In another embodiment, the particles are of any other size suitable for pharmaceutical administration. Each possibility represents a separate embodiment of the present invention.

### Methods of Making Matrix Compositions of the Present Invention

The present invention further provides a process for producing a matrix composition for controlled release of a peptidic molecule comprising:

(a) mixing into a first solvent (i) a biocompatible polymer and (ii) a first lipid component comprising at least one lipid having a polar group, wherein said first solvent is a volatile organic solvent;

(b) mixing the peptidic molecule into a second solvent to form a solution and adding polyethylene glycol into the solution;

(c) mixing the solution obtained in step (b) with a second lipid component comprising at least one phospholipid having fatty acid moieties of at least 14 carbons;

(d) mixing the solutions obtained in steps (a) and (c) to form a homogeneous mixture; and

(e) removing the solvents,

thereby producing a homogeneous polymer-phospholipids matrix comprising the peptidic molecule.

According to certain embodiments, the second solvent is selected from the group consisting of volatile organic solvent and a polar solvent. According to typical embodiments, the polar solvent is water.

According to certain typical embodiments, the method comprises the steps of (a) mixing into a first solvent, preferably a volatile organic solvent: (i) a biodegradable polyester and (ii) sterol; (b) mixing into a different container containing the peptidic molecule dissolved in a second volatile organic solvent or in water and polyethylene glycol (1) a phosphatidylcholine in a second volatile organic solvent and/or (2) a phosphatidylethanolamine in the volatile organic solvent and (3) mixing the resulted solution in a given temperature (4) optionally precipitating the resulted material by centrifugation or by freeze-drying and optionally re-suspending the precipitate in a selected volatile solvent; and (c) mixing and homogenizing the products resulting from steps (a) and (b).

According to certain embodiments, the biodegradable polymer is selected from the group consisting of PLGA, PGA, PLA, chitosan, collagen or combinations thereof. According to some embodiments, the collagen can be any natural or synthetic collagen, for example, bovine collagen, human collagen, a collagen derivative, marine collagen, recombinant or otherwise man made collagens or derivatives or modified versions thereof (e.g. gelatin). Collagen may be of any native or denatured phenotypes such as type I, II, III or IV. In other embodiments, the biodegradable polyester is any other suitable biodegradable polyester known in the art. According to yet additional embodiments, the biodegradable polymer is a polyamine. Mixing the polymer with the at least one lipid having a polar group (non-limiting example being sterol, particularly cholesterol), within the first organic solvent, is typically performed at room temperature. Optionally, α- and/or γ-tocopherol are added to the solution. A lipid-polymer matrix is formed.

The solution containing the at least one peptidic molecule and polyethylene glycol is mixed, typically under stirring, with a volatile organic solvent (selected from the group consisting of, but not limited to N-methylpyrrolidone, ethanol, methanol, ethyl acetate or combination thereof) comprising the at least one phospholipid. According to certain embodiments, the phospholipid is phosphocholine or phosphatidylcholine or derivatives thereof. According to other embodiments, the phospholipid is phosphatidylethanolamine or a derivative thereof. According to additional embodiments, the second volatile organic solvent comprises combination of phosphatidylcholine, phosphatidylethanolamine or derivatives thereof. According to certain embodiments, the phosphocholine or phosphatidylcholine or derivatives thereof is present at 10-90% mass of all lipids in the matrix, i.e. 10-90 mass % of phospholipids, sterols, ceramides, fatty acids etc. According to other embodiments, the phosphatidylethanolamine is present as 10-90 mass % of all lipids in the matrix.

According to yet other embodiments, phosphocholine or phosphatidylcholine derivative or their combination at different ratios with phosphatidylethanolamine are mixed in the organic solvent prior to its addition to the solution comprising the peptide, polypeptide or protein and PEG.

In another embodiment, the phosphatidylethanolamine is also included in the first lipid component.

In another embodiment, the mixture (a) containing the biocompatible polymer is homogenized prior to mixing it with the mixture containing the peptidic molecule and PEG. In another embodiment, the polymer in the mixture of step (a) is lipid saturated. In another embodiment, the matrix composition is lipid saturated. Typically, the polymer and the phosphatidylcholine are incorporated into the matrix composition. In another embodiment, the active peptidic molecule is incorporated into the matrix composition as well. In another embodiment, the matrix composition is in the form of a lipid-saturated matrix whose shape and boundaries are determined by the biodegradable polymer. Each possibility represents a separate embodiment of the present invention.

In another embodiment, the phosphatidylethanolamine has saturated fatty acid moieties. In another embodiment, the fatty acid moieties have at least 14 carbon atoms. In another embodiment, the fatty acid moieties have 14-18 carbon atoms. Each possibility represents a separate embodiment of the present invention.

In another embodiment, the phosphatidylcholine has saturated fatty acid moieties. In another embodiment, the fatty acid moieties have at least 14 carbon atoms. In another embodiment, the fatty acid moieties have at least 16 carbon atoms. In another embodiment, the fatty acid moieties have 14-18 carbon atoms. In another embodiment, the fatty acid moieties have 16-18 carbon atoms. Each possibility represents a separate embodiment of the present invention.

In another embodiment, the molar ratio of total lipids to polymer in the non-polar organic solvent is such that the polymer in this mixture is lipid-saturated. In another embodiment for purposes of illustration, in the case wherein the polymer is predominantly 50 KDa PLGA (poly(lactic-co-glycolic acid, 1:1 ratio)), the molar ratio of total lipids to 50 KDa PLGA is typically in the range of 10-50 inclusive. In another embodiment, the molar ratio of total lipids to 50 KDa PLGA is between 10-100 inclusive. In another embodiment, the molar ratio is between 20-200 inclusive. In another embodiment, the molar ratio is between 20-300 inclusive. In another embodiment, the molar ratio is between 30-400 inclusive. Each possibility represents a separate embodiment of the present invention.

Each of the components of the above method and other methods of the present invention is defined in the same manner as the corresponding component of the matrix compositions of the present invention.

In another embodiment, step (a) of the production method further comprises adding to the volatile organic solvent, typically non-polar solvent, a phosphatidylethanolamine. In another embodiment, the phosphatidylethanolamine is the same phosphatidylethanolamine included in step (c). In another embodiment, the phosphatidylethanolamine is a different phosphatidylethanolamine that may be any other phosphatidylethanolamine known in the art. In another embodiment, the phosphatidylethanolamine is selected from the group consisting of the phosphatidylethanolamine of step (c) and a different phosphatidylethanolamine. Each possibility represents a separate embodiment of the present invention.

In another embodiment, step (c) of the production method further comprises adding to the solvent, typically a volatile organic solvent, more typically a water-miscible solvent, a phospholipid selected from the group consisting of a phosphatidylserine, a phosphatidylglycerol, a sphingomyelin, and a phosphatidylinositol.

In another embodiment, step (c) of the production method further comprises adding to the water-miscible volatile organic solvent a sphingolipid. In another embodiment, the sphingolipid is ceramide. In another embodiment, the sphingolipid is a sphingomyelin. In another embodiment, the sphingolipid is any other sphingolipid known in the art. Each possibility represents a separate embodiment of the present invention.

In another embodiment, step (c) of the production method further comprises adding to the water-miscible, volatile organic solvent an omega-6 or omega-9 free fatty acid. In another embodiment, the free fatty acid has 16 or more carbon atoms. Each possibility represents a separate embodiment of the present invention.

Upon mixing, a homogenous mixture is formed, since the polymer is lipid-saturated in the mixture of step (a). In another embodiment, the homogenous mixture takes the form of a homogenous liquid. In another embodiment, upon freeze-drying or spray-drying the mixture, vesicles are formed. Each possibility represents a separate embodiment of the present invention.

In another embodiment, the production method further comprises the step of removing the solvent and optionally water present in the product of step (d). In certain embodiments, the solvent and water removal utilizes atomization of the mixture. In other embodiments, the mixture is atomized into dry, heated air. Typically, atomization into heated air evaporates all solvents and water immediately, obviating the need for a subsequent drying step. In another embodiment, the mixture is atomized into a water-free solvent. In another embodiment, the liquid removal is performed by spray drying. In another embodiment, the liquid removal is performed by freeze drying. In another embodiment, the liquid removal is performed using liquid nitrogen. In another embodiment, the liquid removal is performed using liquid nitrogen that has been pre-mixed with ethanol. In another embodiment, the liquid removal is performed using another suitable technique known in the art. Each possibility represents a separate embodiment of the present invention.

In another embodiment, a method of the present invention further comprises the step of vacuum-drying the composition. In another embodiment, the step of vacuum-drying is performed following the step of evaporation. Each possibility represents a separate embodiment of the present invention.

In another embodiment, the method of the present invention further comprises the step of evaporating the solvent by heating the product of step (d). The heating is continuing until the solvent is eliminated and in a typical temperature between room temperature to 90° C., more typically up to 50° C. In another embodiment a step of vacuum-drying is performed following the step of evaporating. Each possibility represents a separate embodiment of the present invention.

The present invention further provides a process for coating a substrate with a matrix composition for controlled release of a peptidic molecule comprising:

(a) mixing into a first solvent (i) a biocompatible polymer and (ii) a first lipid component comprising at least one lipid having a polar group, wherein said first solvent is a volatile organic solvent;

(b) mixing the peptidic molecule into a second solvent to form a solution and adding polyethylene glycol into the solution;

(c) mixing the solution obtained in step (b) with a second lipid component comprising at least one phospholipid having fatty acid moieties of at least 14 carbons;

(d) mixing the solutions obtained in steps (a) and (c) to form a homogeneous mixture;

(e) adding, dipping or immersing a substrate into the homogeneous mixture obtained in step (d) or spraying the substrate with the homogenous mixture obtained in step (d)

(f) removing the solvents from the coated substrates. According to certain embodiments, the substrates to be coated include at least one material selected from the group consisting of carbon fibers, stainless steel, hydroxylapatite coated metals, synthetic polymers, rubbers, silicon, cobalt-chromium, titanium alloy, tantalum, ceramic and collagen or gelatin. In other embodiments substrates may include any medical devices and bone filler particles. Bone filler particles can be any one of allogeneic (i.e., from human sources), xenogeneic (i.e., from animal sources) and artificial bone particles. According to certain typical embodiments, the coating has a thickness of 1-200 μm; preferably between 5-100 μm.

According to some embodiments, the removal of solvents from the coated substrates may be performed by evaporation, for example by placing the coated substrate in an incubator at a temperature of 37° C., or by continuous drying under vacuum, or by applying negative pressure to accelerate the solvent removal. Finally, in some cases, another step of negative pressure is used to remove any residual solvents. The term ‘negative pressure’ as used herein refers to pressure below atmospheric pressure.

### Lipid Saturation and Techniques for Determining Same

“Lipid saturated,” as used herein, refers to saturation of the polymer of the matrix composition with phospholipids in combination with a therapeutic peptidic molecule and optionally targeting moiety present in the matrix, and any other lipids that may be present. As described herein, matrix compositions of the present invention comprise, in some embodiments, phospholipids other than phosphatidylcholine. In other embodiments, the matrix compositions may comprise lipids other than phospholipids. The matrix composition is saturated by whatever lipids are present. “Saturation” refers to a state wherein the matrix contains the maximum amount of lipids of the type utilized that can be incorporated into the matrix. Methods for determining the polymer:lipid ratio to attain lipid saturation and methods of determining the degree of lipid saturation of a matrix are known to a person skilled in the art. Each possibility represents a separate embodiment of the present invention.

According to certain typical embodiments, the final matrix composition of the present invention is substantially free of water in contrast to hitherto known lipid-based matrices designed for the delivery of peptidic molecules, particularly peptides, polypeptides and proteins having therapeutic activity. In other words, even when the active ingredients are initially dissolved in an aqueous solution all the solvents are removed during the process of preparing the lipid polymer compositions. The substantially absence of water from the final composition protects the bioactive peptidic molecule from degradation or chemical modification, particularly from enzyme degradation. Upon application of the composition to a hydrous biological environment, the outer surface of the matrix composition contacts the biological liquids while the substantially water free inner part protects the remaining active ingredient thus enabling sustained release of undamaged active ingredient.

According to certain embodiments, the term “substantially free of water” refers to a composition containing less than 1% water by weight. In another embodiment, the term refers to a composition containing less than 0.8% water by weight. In another embodiment, the term refers to a composition containing less than 0.6% water by weight. In another embodiment, the term refers to a composition containing less than 0.4% water by weight. In another embodiment, the term refers to a composition containing less than 0.2% water by weight. In another embodiment, the term refers to the absence of amounts of water that affect the water-resistant properties of the matrix.

In another embodiment, the matrix composition is essentially free of water. “Essentially free” refers to a composition comprising less than 0.1% water by weight. In another embodiment, the term refers to a composition comprising less than 0.08% water by weight. In another embodiment, the term refers to a composition comprising less than 0.06% water by weight. In another embodiment, the term refers to a composition comprising less than 0.04% water by weight. In another embodiment, the term refers to a composition comprising less than 0.02% water by weight. In another embodiment, the term refers to a composition comprising less than 0.01% water by weight. Each possibility represents a separate embodiment of the present invention.

In another embodiment, the matrix composition is free of water. In another embodiment, the term refers to a composition not containing detectable amounts of water. Each possibility represents a separate embodiment of the present invention.

The process of preparing the matrix of the present invention comprises only one step where an aqueous solution may be used. This solution is mixed with organic volatile solvent, and all the liquids are removed thereafter. The process of the present invention thus enables lipid saturation. Lipid saturation confers upon the matrix composition ability to resist bulk degradation in vivo; thus, the matrix composition exhibits the ability to mediate extended release on a scale of several weeks or months.

In another embodiment, the matrix composition is dry. “Dry” refers, in another embodiment, to the absence of detectable amounts of water or organic solvent.

In another embodiment, the water permeability of the matrix composition has been minimized. “Minimizing” the water permeability refers to a process of producing the matrix composition mainly in organic solvents, as described herein, in the presence of the amount of lipid that has been determined to minimize the permeability to penetration of added water. The amount of lipid required can be determined by hydrating the vesicles with a solution containing tritium-tagged water, as described herein.

In another embodiment, “lipid saturation” refers to filling of internal gaps (free volume) within the lipid matrix as defined by the external border of the polymeric backbone. The gaps are filled with the phospholipids in combination with any other types of lipids, peptidic molecule and optionally targeting moiety present in the matrix, to the extent that additional lipid moieties can no longer be incorporated into the matrix to an appreciable extent.

Zero-order release rate” or “zero order release kinetics” means a constant, linear, continuous, sustained and controlled release rate of the bioactive peptidic molecule from the polymer matrix, i.e. the plot of amounts of the peptidic molecule released vs. time is linear.

### Therapeutic Applications of the Bioactive Peptidic Molecule

The present invention also relates to a variety of applications, in which a sustained or controlled release of a pharmaceutically active peptidic molecule is desired. Thus, according to certain embodiments, the present invention provides a method of administering at least one type of a therapeutically effective peptidic molecule to a subject in need thereof, the method comprising the step of administering to the subject a pharmaceutical composition of the present invention, thereby administering the at least one peptidic molecule to the subject.

According to certain typical embodiments, the present invention provides a method of administering at least one type anti-microbial peptide to a subject in need thereof, the method comprising the step of administering to the subject a pharmaceutical composition

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1

### Platform Technology for Production of Drug Carrier Compositions for the Delivery of Peptidic Molecules

### I. Preparation of First Solution

A Polymer (for example, PLGA, PGA, PLA, or a combination thereof) and a sterol (e.g. cholesterol) and/or alpha- or gamma tocopherol are mixed in a volatile organic solvent (e.g. ethyl acetate with/without chloroform). The entire process is performed at room temperature. A lipid-polymer matrix is thus obtained.

### II. Preparation of Second Solution

At least one molecule selected from a peptide, a protein or any combination thereof is dissolved in a volatile organic solvent (typically N-methylpyrrolidone, ethanol, methanol, ethyl acetate or combination thereof) or water and polyethylene glycol (PEG) 1,000-8000, typically PEG 5,000 is added. When the peptidic molecule is dissolved in organic solvent, a phospholipid is added directly. When the peptidic molecule is dissolved in water, the resulted solution is mixed, typically under stirring, with a volatile organic solvent (typically N-methylpyrrolidone, ethanol, methanol, ethyl acetate or combination thereof) comprising the phospholipd. The added phospholipid comprises:

A phosphocholine or phosphatidylcholine derivative, e.g. deuterated 1,2-distearoyl-sn-glycero-3-phosphocholine (DSPC) or dioleoyl-phosphatidylcholine (DOPC), Dipalmitoyl-phosphatidylcholine (DPPC), Dimyristoyl-phosphatidylcholine (DMPC), dioleoyl-phosphatidylcholine (DOPC), 1-palmitoyl-2-oleoyl-phosphatidylcholine, present as 10-90 mass % of all lipids in the matrix, i.e. 10-90 mass % of phospholipids, sterols, ceramides, fatty acids etc;

Optionally, phosphatidylethanolamine—e.g. dimethyldimyristoyl phosphatidylethanolamine (DMPE) or dipalmitoyl-phosphatidylethanolamine (DPPE)—present as 10-90 mass % of all lipids in the matrix;

Optionally, phosphocholine or phosphatidylcholine derivative or their combination at different ratios of phosphatidylethanolamine, mixed in the organic solvent prior to its addition of the NA drug water based solution;

Optionally, cationic lipid is included as 0.1-10 mol % of all lipids in the matrix;

Optionally, 0.1-15 mass % of a free fatty acid, e.g. linoleic acid (LN), or oleic acid (OA), as 0.1-10 mass % of all lipids in the matrix;

The mixture is homogenized, sonicated or used for coating the surface of medical devices. Typically the entire process is conducted at room temperature and up to 50° C.

III. Mixing the Polymer with the Peptidic Molecule-PEG Mixture

The second suspension (or solution) is added to the first solution under stirring. Stirring is continued for up to about 5 h. The entire process is performed preferably at room temperature, with heating if necessary preferably to no more than 60° C., but in any case at a temperature which does not cause denaturation of the peptidic molecule, all according to the specific formulation, the nature of the lipids in use and the specific peptidic molecule. The resulting mixture should be homogenous, but can also be slightly turbid.

### IV. Removal of the Solvents

When coating of surfaces is performed; the suspension from stage III is mixed with the particles or devices to be coated followed by evaporation of the volatile organic solvents. The entire coating process is performed at a temperature of about 30-60° C., typically about 45° C.

The volatile organic solvents may be optionally be removed by evaporation by placing the coated substrate in an incubator at a temperature of 37° C., or by continuous drying under vacuum, or by applying negative pressure to accelerate the solvent removal.

The solution from stage III may be optionally atomized into dry, heated air.

Alternatively the solution from stage III is atomized into water based solution, which may contain carbohydrates, or atomized into ethanol covered by liquid nitrogen or only liquid nitrogen without ethanol, after which the nitrogen and/or ethanol (as above) are evaporated.

### V. Vacuum Drying

The matrix composition, coated particles and coated devices are vacuum-dried. All organic solvent and water residues are removed. The lipid-based matrix comprising the peptidic molecule is ready for storage.

### Example 2

### Preparation of a Matrix Comprising Anti-Microbial Peptide without PEG

The anti-microbial peptide used was Temporin-L (SEQ: FVQWFSKFLGRIL) labeled with the fluorescent dye NBD at its N-terminal.
1. The peptide (1 mg) was dissolved in MeOH/EA and this solution was used in order to produce a matrix formulation without PEG.
2. DPPC was dissolved into the peptide solution to final concentration of 225 mg/ml.
3. PLGA 75/25 was dissolve in ethyl acetate (300 mg/ml).
4. Cholesterol was dissolve in ethyl acetate (30 mg/ml).
5. One volume of the PLGA solution was mixed with 5 volumes of the cholesterol solution.
6. Two volumes of the DPPC-peptide solution were mixed with three volumes of the PLGA-cholesterol solution.
7. 100 mg of tricalcium phosphate particles (TCP) were weighed into 4 ml glass vial.
8. 0.15 ml of the PLGA-cholesterol-DPPC-peptide solution was added to the TCP particles. The resulting solution was incubated at 45° C. until all solvents evaporated; any remaining solvents were discarded by overnight vacuum.

### Example 3

### Preparation of a Matrix Comprising Anti-Microbial Peptide with PEG

1. The peptide was dissolved in MeOH/EA as in Example 2 above.
2. 0.5 mg of PEG 8,000 was dissolved into the peptide solution of step 1.
3. The solution was incubated at 45° C. for 10 minutes
4. DMPC or DPPC were dissolved in the peptide-PEG solution (final phospholipids concentration 225 mg/ml).
5. PLGA 75/25 was dissolved in ethyl acetate (300 mg/ml).
6. Cholesterol (30 mg/ml) was dissolved in ethyl acetate.
7. One volume of the PLGA solution was mixed with 5 volumes of the cholesterol solution.
8. Two volumes of either DPPC-PEG-peptide or DMPC-PEG-peptide solution were mixed with three volumes of the PLGA-cholesterol solution.
9. 200 mg TCP were weighed into 4 ml glass vials.
10. 0.2 ml of the PLGA-cholesterol-DPPC-PEG-peptide solution or the PLGA-cholesterol-DMPC-PEG-peptide solution was added to the TCP particles.
11. The resulted solution was incubated at 45° C. until all solvents evaporate; any remaining solvents were discarded by overnight vacuum.

### Example 4

### Release of the Peptide from the Formulation

The bone graft TCP (Tricalcium Phosphate) coated with matrix composition comprising the anti-microbial peptide Temporin-L was hydrated by 0.2 ml of double distilled water (DDW) and samples were daily collected by replacing the supernatant with a fresh new volume of supernatant. The peptide was extracted by adding one volume of MeOH to one volume of sample, vortex, and centrifugation for 2 min 16000 rpm. The supernatant was then diluted two-fold in MeOH/DDW.

The amount of the anti-microbial peptide released to the solution was evaluated by following the fluorescence of NBD (Ex 485 nm, Em 520 nm). The results, plotted against linear standard curve derived from the fluorescence intensity of two fold serial dilutions of the peptide in ddw/MeOH are presented in FIG. 1. The results clearly demonstrate that addition of polyethylene glycol to the matrix improved significantly the period and rate of the protein release.

### Example 5

### Sustained Release of Fibroblast Growth Factor (FGF) from Bone Filler Coated with the Matrix Composition According to Some Embodiments of the Invention

Bone filler particles coated with a matrix composition comprising FGF (human FGF-2 Sigma) with and without PEG were prepared as described above in Examples 2 and 3. In this matrix composition the phospholipids were successfully dissolved in a mixture of methanol and ethyl acetate and only then 1 volume of FGF solution with or without PEG was mixed with 10 volumes of the phospholipids solution.

Samples of the coated bone filler particles were hydrated with DDW in order to initiate the release of FGF from the matrix composition. The solution in the samples was replaced and collected daily and was kept at 4° C. until analysis.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A matrix composition comprising:
a. a biocompatible polymer in association with a first lipid component comprising cholesterol;
b. a second lipid component comprising at least one phospholipid having fatty acid moieties of at least 14 carbons, said phospholipid being selected from the group consisting of (i) phosphatidylcholine or a derivative thereof, (ii) a mixture of phosphatidylcholines or derivatives thereof, (iii) a phosphatidylethanolamine or a derivative thereof, and any combination of (i), (ii) and (iii); and
c. at least one peptidic molecule in association with polyethylene glycol (PEG);
wherein the matrix composition is adapted for providing sustained and/or controlled release of the peptidic molecule, and wherein the weight ratio of the peptidic molecule and PEG is between 10:1 and 1:1 inclusive.

2. The matrix composition of claim 1, wherein the peptidic molecule is polar.

3. The matrix composition of claim 1, wherein the PEG is a linear PEG having a molecular weight in the range of 1,000-8,000.

4-6. (canceled)

7. The matrix composition of claim 1, wherein the cholesterol is present in an amount of 2-30 mole percent of the total lipid content of said matrix composition.

8. (canceled)

9. The matrix composition of claim 1, wherein the phospholipid comprises at least one saturated fatty acid moiety of at least 14 carbons.

10. The matrix composition of claim 8, wherein the phospholipid comprises two saturated fatty acid moieties of at least 14 carbons.

11. The matrix composition of claim 1, further comprising a cationic lipid selected from the group consisting of DC-Cholesterol, 1,2-dioleoyl-3-trimethylammonium-propane (DOTAP), Dimethyldioctadecylammonium (DDAB), 1,2-dilauroyl-sn-glycero-3-ethylphosphocholine (Ethyl PC), 1,2-di-O-octadecenyl-3-trimethylammonium propane (DOTMA) and any combination thereof.

12. The matrix composition of claim 1, wherein the biocompatible polymer is selected from the group consisting biodegradable polymer, non-biodegradable polymer and a combination thereof.

13. The matrix composition of claim 12, wherein the biodegradable polymer is a selected from the group consisting of PLA (polylactic acid), PGA (poly glycolic acid) PLGA (Poly(lactic co glycolic acid), chitosan, collagen and its derivatives and combinations thereof.

14. The matrix composition of claim 13, wherein the non-biodegradable polymer is selected from the group consisting of, PEG acrylate, PEG methacrylate, methylmethacrylate, ethylmethacrylate, butylmethacrylate, 2-ethylhexylmethacrylate, laurylmethacrylate, hydroxylethyl methacrylate, 2-methacryloyloxyethylphosphorylcholine (MPC), polystyrene, derivatized polystyrene, polylysine, poly N-ethyl-4-vinyl-pyridinium bromide, poly-methylacrylate, silicone, polyoxymethylene, polyurethane, polyamides, polypropylene, polyvinyl chloride, polymethacrylic acid and combination thereof.

15. The matrix composition of claim 14, wherein the biocompatible polymer comprises co-block of a biodegradable polymer and a non-biodegradable polymer.

16. The matrix composition of claim 1, wherein the weight ratio of total lipids to the biodegradable polymer is between 1:1 and 9:1 inclusive.

17. (canceled)

18. The matrix composition of claim 1, wherein said matrix composition is homogeneous.

19-21. (canceled)

22. The matrix composition of claim 1, further comprising an additional phospholipid selected from the group consisting of a phosphatidylserine, a phosphatidylglycerol, and a phosphatidylinositol.

23. The matrix composition of claim 1, further comprising a free fatty acid having 14 or more carbon atoms.

24. The matrix composition of claim 1, further comprising a PEGylated lipid.

25. The matrix composition of claim 1, wherein when hydrated at least 30% of said peptidic molecule is released from the composition at zero-order kinetics.

26. (canceled)

27. The matrix composition of claim 1 wherein the peptidic molecule has a therapeutic activity.

28. (canceled)

29. The matrix composition of claim 28, wherein the peptidic molecule is anti-microbial peptide.

30-31. (canceled)

32. The matrix composition of claim 1, said matrix comprises (a) biodegradable polyester; (b) a sterol; (c) a phosphatidylcholine having fatty acid moieties of at least 14 carbons; (d) a polar peptidic molecule; and (e) PEG.

33-35. (canceled)

36. A medical device, comprising: a substrate and a biocompatible coating deposited on at least a fraction of said substrate, wherein said biocompatible coating comprises the matrix composition of claim 1.

37. (canceled)

38. A method of producing a matrix composition for delivery and sustained and/or controlled release of a peptidic molecule comprising the steps of:
a. mixing into a first solvent (i) a biocompatible polymer and (ii) a first lipid component comprising cholesterol; wherein said first solvent is a volatile organic solvent;
b. mixing the peptidic molecule into a second solvent to form a solution and adding polyethylene glycol into the solution;
c. mixing the solution obtained in step (b) with a second lipid component comprising at least one phospholipid having fatty acid moieties of at least 14 carbons, said phospholipid being selected from the group consisting of (i) phosphatidylcholine or a derivative thereof, (ii) a mixture of phosphatidylcholines or derivatives thereof, (iii) a phosphatidylethanolamine or a derivative thereof, and any combination of (i), (ii) and (iii);
d. mixing the solutions obtained in steps (a) and (c) to form a homogeneous mixture; and
e. removing the solvents by heating to no more than 60° C.;
thereby producing a homogeneous polymer-phospholipids matrix comprising the peptidic molecule.

39. The method of claim 38, wherein the second solvent is selected from the group consisting of volatile organic solvent and a polar solvent.

40-51. (canceled)


SEQUENCE LISTING (2 sequences)

SEQ ID NO: 1 - protein, 13 aa, Artificial
  Chemically synthesized
FVQWFSKFLGRIL

SEQ ID NO: 2 - protein, 288 aa, HOMO_SAPIENS
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS